(19) United States
(12) Patent Application Publication
(10) Pub. No.: US 2025/0000943 A1
(43) Pub. Date: Jan. 2, 2025

(54) MODULATORS OF THE G3BP2-TAU INTERACTION FOR THE TREATMENT OF TAU ASSOCIATED DISEASES

(71) Applicant: Hoffmann-La Roche Inc., Little Falls, NJ (US)

(72) Inventors: Ravi JAGASIA, Riehen (CH); Marco TERRIGNO, Basel (CH); Congwei WANG, Nærum (DK)

(73) Assignee: Hoffmann-La Roche Inc., Little Falls, NJ (US)

(21) Appl. No.: 18/883,958

(22) Filed: Sep. 12, 2024

Related U.S. Application Data
(63) Continuation of application No. PCT/EP2023/056281, filed on Mar. 13, 2023.

(30) Foreign Application Priority Data
Mar. 14, 2022 (EP) 22161861.4

(51) Int. Cl. A61K 38/17

(52) U.S. Cl. CPC A61K 38/1709

(57) ABSTRACT

Provided are methods for the treatment of a Tau associated disease, comprising administering to a subject an effective amount of a compound modulating a protein-protein interaction between a protein comprising a NTF2-like domain having the amino acid sequence set forth as SEQ ID NO: 3 and Tau protein.

### CROSS REFERENCE TO RELATED APPLICATIONS

This application is a continuation of International Patent Application No. PCT/EP2023/056281, filed Mar. 13, 2023, which claims priority to European Patent Application No. 22161861.4 filed Mar. 14, 2022, which are incorporated herein by reference in their entirety.

### SEQUENCE LISTING

This application contains a Sequence Listing which has been submitted electronically in XML format and is hereby incorporated by reference in its entirety. Said XML copy, created on Aug. 15, 2024, is named P37430-US Sequence Listing and is 8,946 bytes in size.

### FIELD OF THE INVENTION

The present invention relates to methods for the treatment of Tau associated diseases.

### BACKGROUND

The toxic accumulation of the microtubule-associated protein Tau is a hallmark of multiple neurodegenerative diseases, including Alzheimer's disease (AD), frontotemporal dementia with parkinsonism-17 (FTDP-17), progressive supranuclear palsy (PSP), corticobasal degeneration (CBD) and Pick's disease (PiD); these diseases are collectively known as Tauopathies. In the healthy brain, Tau is involved in microtubule assembly and stabilization, while under disease conditions, hyperphosphorylated Tau detaches from microtubules and further aggregates to form paired helical filaments (PHFs) and neurofibrillary tangles (NFTs). The currently limited understanding of the mechanisms underlying Tauopathies impedes the development of effective treatments for these diseases.

Therefore, there is a need for the identification of new target proteins and their use for the treatment of Tauopathies.

### DESCRIPTION OF THE INVENTION

In a first object, the present invention provides a method for the treatment of a Tau associated disease comprising administering to a subject an effective amount of a compound modulating the protein-protein interaction between a protein comprising a NTF2-like domain having an amino acid sequence set forth in SEQ ID NO: 3 or variants thereof and Tau protein.

In an embodiment, the compound is promoting/enhancing the protein—protein interaction between the protein comprising the NTF2-like domain and Tau protein.

In an embodiment, the compound is a mimetic of the protein—protein interaction between the protein comprising a NTF2-like domain and Tau protein.

In an embodiment, the Tau associated disease is selected from the group consisting of Alzheimer's Disease, amyotrophic lateral sclerosis, Parkinson's disease, Dementia pugilistica, Down's Syndrome, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotemporal dementia, frontotemporal dementia with parkinsonism linked to chromosome 17, Hallevorden-Spatz disease, Niemann-Pick disease type C, Pallido-ponto-nigral degeneration, Pick's disease, progressive subcortical gliosis, progressive supranuclear palsy, tangle-only dementia, postencephalitic Parkinsonism, and myotonic dystrophy.

In an embodiment, the protein comprising the NTF2-like domain is a G3PB2 protein.

In an embodiment, the protein comprising the NTF2-like domain and the Tau protein are human proteins.

In a second object, the present invention provides a compound modulating the protein-protein interaction between a protein comprising a NTF2-like domain having an amino acid sequence set forth in SEQ ID NO: 3 or variants thereof and Tau protein for use in the treatment of a Tau associated disease.

In an embodiment of the second object, the Tau associated disease is selected from the group consisting of Alzheimer's Disease, amyotrophic lateral sclerosis, Parkinson's disease, Dementia pugilistica, Down's Syndrome, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotemporal dementia, frontotemporal dementia with parkinsonism linked to chromosome 17, Hallevorden-Spatz disease, Niemann-Pick disease type C, Pallido-ponto-nigral degeneration, Pick's disease, progressive subcortical gliosis, progressive supranuclear palsy, tangle-only dementia, postencephalitic Parkinsonism, and myotonic dystrophy.

In a third object, the present invention provides a protein comprising a NTF2-like domain having an amino acid sequence set forth in SEQ ID NO: 3 or variants thereof for use in the identification of a compound modulating the protein — protein interaction between the protein comprising the NTF2-like domain and Tau protein.

In a fourth object, the present invention provides a protein comprising a NTF2-like domain having an amino acid sequence set forth in SEQ ID NO: 3 or variants thereof for use as a target protein for the treatment of a Tau associated disease.

In an embodiment of the third and fourth object, the protein is human G3BP2 protein.

### BRIEF DESCRIPTION OF THE DRAWINGS

FIGS. 1A and 1B: G3BP2 directly interacts with Tau and inhibits Tau aggregation in vitro.

FIG. 1A) SPR sensorgrams and binding affinity (Kd) of full length Tau interacting with full length G3BP2. Tau was immobilized on the sensor chip and G3BP2 was injected at corresponding concentrations.

FIGS. 1B and 1C) Kinetics (FIG. 1B) and barplot (FIG. 1C) showing the effect of G3BP2 on Tau aggregation (n=6). Error bars: mean±SD. The p-values were obtained using one-way ANOVA followed by Dunnett's multiple comparison test (post hoc test) compared to control.

FIGS. 2A and 2B: Aggregated Tau species is increased upon G3BP2 knockdown via Tau seeding assay in hiPSC-derived neurons.

FIG. 2A) Schematic illustration of Tau seeding assay in hiPSC-derived neurons. OE: overexpression. Tau PFFs: Tau preformed fibrils.

FIG. 2B) Quantification of aggregated Tau species in the Tau seeding assay. The p-value was obtained using a two-tailed, unpaired Student's t test.

FIGS. 3A-3C: Tau pathology is elevated in the absence of G3BP2.

FIG. 3A) Immunohistochemistry of phospho-Tau (green) and total Tau (magenta) in 4-month-old fAD (PSEN1 M139V) or fAD G3BP2 knockout human cerebral organoids. Nuclei stained with DAPI in blue. Scale bar: 50 μm.

FIG. 3B) Whole-slide image displaying the IHC of Tau pS214 (green), total Tau (yellow) and MAP2 (red). Scale bar: 2 mm.

FIG. 3C) Quantification of Tau pT181 and pT231 by ELISA. pTau levels were normalized to total Tau levels. Each data point represents 4-5 pooled hCGs. Error bars: mean±SD. The p-values were obtained using two-tailed, unpaired Student's t-tests.

FIGS. 4A and 4B: Progressively increasing G3BP2—Tau interaction in the human AD brain.

FIG. 4A) Representative images of PLA signal (green) showing G3BP2 interacting with Tau in Braak I, IV and VI AD cases. Total Tau was stained with Tau polyclonal antibody (magenta). Nuclei stained with DAPI (blue). Scale bar: 10 μm.

FIG. 4B) PLA puncta of G3BP2-Tau per cell were quantified in patients with Braak I (n=9), II&III (n=10), IV&V (n=10) and VI (n=9) AD. For boxplots, median, 25th and 75th percentiles are shown. The p-value was obtained using two-tailed, unpaired Student's t-tests. PLA: proximity ligation assay. AD: Alzheimer's disease.

FIGS. 5A-5D: The NTF2-like domain of G3BP2 can inhibit Tau aggregation.

FIG. 5A) Diagram of the amino acid sequence of full-length G3BP2.

FIG. 5B) Amino acid sequence of G3BP2's NTF2-like domain (SEQ ID NO: 3).

FIGS. 5C and 5D) Kinetics (FIG. 5C) and bar plot (FIG. 5D) showing the effect of the G3BP2 NTF2-like domain on Tau aggregation (n=5). The data are shown as the mean±SD. The p-values compared to the control group were obtained using one-way ANOVA followed by Dunnett's multiple comparison test (post hoc test).

### DEFINITIONS

An “individual” or “subject” is a mammal Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

Tau protein is normally associated with microtubules and plays a crucial function in their assembly, stabilizing microtubules against dynamic instability and facilitating microtubule binding to other cytoskeletal filaments. (Mactui R. B. and Change V. (1995) Role of microtubule-associated proteins in the control of microtubule assembly. Physiol Rev. 75 (4):835-864; Mactui, R. B., Barbeita L, and Mutioz J. P. (20001). The molecular bases of Alzheimer's disease and other neurodegenerative disorders. Arch. Medical Research. 32-367-381). Tau protein belongs to the family of MAPs, or microtubule-associated proteins. In humans, it is found almost exclusively in neurons (Mactui R. B. and Arechaga J. (1987). “The Cytoskeleton in Cell Differentiation and Development”. Oxford University Press, U.K. 367 pp; Mactui R. B. and Change V. (1995.) Role of microtubule-associated proteins in the control of microtubule assembly. Physiol Rev. 75 (4):835-864) and occurs in 6 isoforms, which derive from the expression of a single gene. This gene is found on the long arm of chromosome 17. at position 21 (17q21) and contains 13 exons, which by an alternative splicing process generate 6 molecular isoforms, which have between 352 and 441 amino acids (Goedert M. (2004). Tau protein and neurodegeneration. Seminars in Cell & Developmental Biology. 15:45-49).

Exemplary Tau protein associated diseases or disorders include, without limitation, Alzheimer's Disease, amyotrophic lateral sclerosis, Parkinson's disease, Dementia pugilistica, Down's Syndrome, traumatic brain injury, amyotrophic lateral sclerosis/parkinsonism-dementia complex of Guam, Non-Guamanian motor neuron disease with neurofibrillary tangles, argyrophilic grain dementia, cortico-basal degeneration, diffuse neurofibrillary tangles with calcification, frontotemporal dementia, fronto-temporal dementia with parkinsonism linked to chromosome 17, Hallevorden-Spatz disease, Niemann-Pick disease type C, Pallido-ponto-nigral degeneration, Pick's disease, progressive subcortical gliosis, progressive supranuclear palsy, tangle-only dementia, postencephalitic Parkinsonism, and myotonic dystrophy.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

Herein, “G3BP2” is an abbreviation used for Ras GTPase-activating protein-binding protein 2. Isoform A of human G3BP2 has the Uniprot ID: Q9UN86-1 (SEQ ID NO: 1) and Isoform B of human G3BP2 has the Uniprot ID: Q9UN86-2 (SEQ ID NO: 2). Furthermore, variant G3BP2 proteins herein also include functional fragments or derivatives thereof.

The term “NTF2-like domain” as used herein refers to a protein domain comprising the amino acid sequence set forth in SEQ ID NO: 3 and this term encompasses NTF2-like domain variants having at least 80%, 85%, 90%, 95% and 100% amino acid sequence identity with the NTF2-like domain amino acid sequence set forth in SEQ ID NO: 3. The NTF2-like domain variants can have a shorter amino acid sequence length, the same amino acid sequence length or a longer amino acid sequence length than the amino acid sequence set forth in SEQ ID NO: 3 provided that these NTF2-like domain variants retain their ability to interact with Tau protein (functional NTF2-like domain variants). NTF2 refers to Nuclear transport factor 2 protein.

The terms “modulator” and “inhibitor” as used herein refer to compounds, which reversibly or irreversibly promote/enhance and/or inhibit the protein—protein interaction between a protein comprising a NTF2-like domain and Tau protein.

The term “mimetic” as used herein refers to compounds that structurally mimic the natural protein—protein interaction between a protein comprising a NTF2-like domain and Tau protein.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein: protein) program and default options (BLOSUM50; open: -10; ext:-2; Ktup=2) to ensure a global, rather than local, alignment is performed. Percent amino acid identity is given in the output alignment header.

### Material & Methods

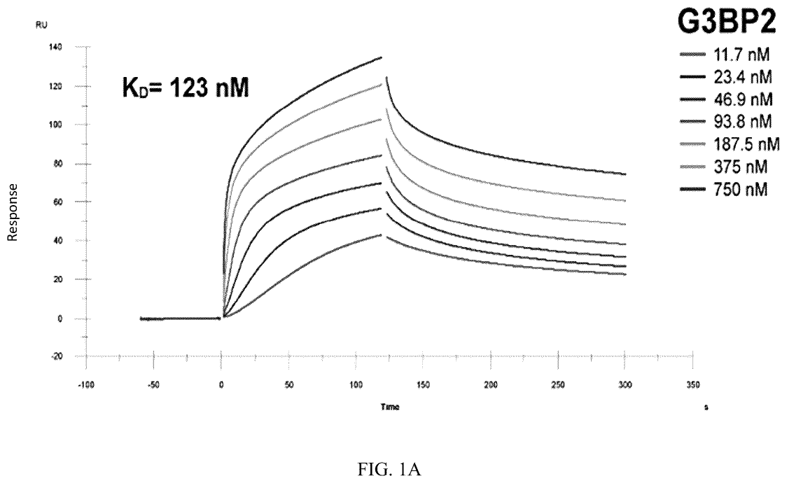

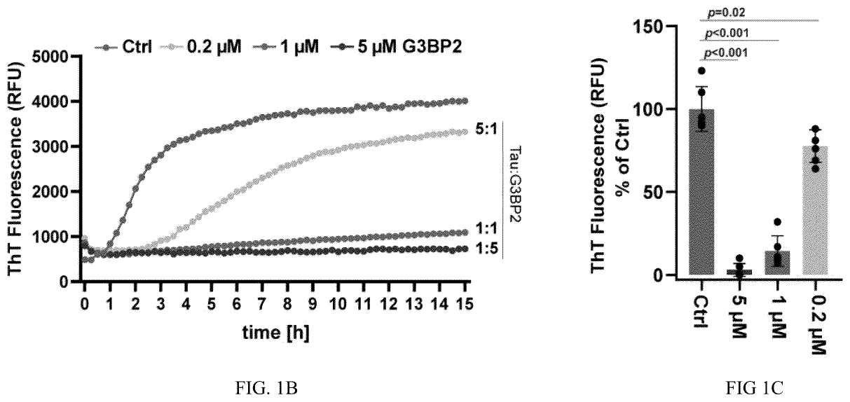

### FIG. 1A:

Surface plasmon resonance (SPR) experiment was performed in a Biacore T200 (Cytiva) equipped with Biacore™ T200 Control Software (V2.0.2, Cytiva) at 25° C. with HBS-EP+ as running buffer (Cytiva, Cat. BR100669) at Agro-Bio (La Ferté-Saint-Aubin, France). The following recombinant proteins were used in SPR. Active Human Recombinant Tau441 (2N4R) wild-type protein monomers (StressMarq, Cat. SPR-479) and customized His tagged human full length G3BP2 (1-449) was produced at GenScript Biotech (Piscataway, USA).

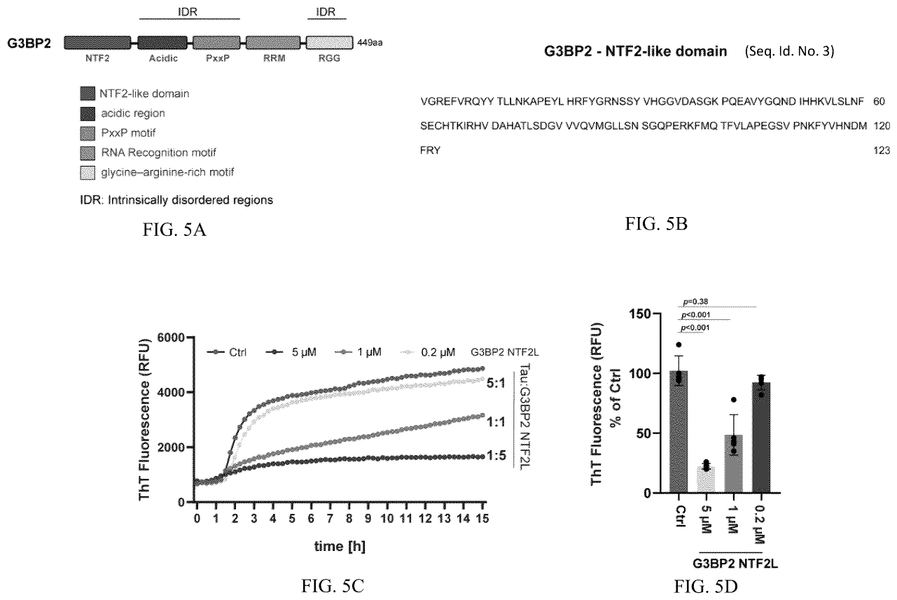

### FIGS. 1B, 5C and 5D:

To mimic Tau aggregation in vitro, recombinant Tau441 (2N4R) P301L (Analytik Jena, Cat. T-1014-1) at 1 μM final concentration, was incubated with 30 μM sodium octadecylsulfate (ODS) and 1 μM heparin in reagent buffer (20 μM Thioflavine T, 5 mM 1,4-dithioerythreitol (DTT), 100 mM sodium chloride, 10 mM HEPES pH 7.4) for 15 h at 37° C. in black no-binding 96 well plates. G3BP2 at 3 concentrations (0.2, 1 and 5 μM) were incubated with Tau-Heparin-ODS-Buffer solution Immediately after preparation, a baseline measurement was carried out and kinetic was monitored every 15 mM for 15 h while incubating at 37° C., by using 450 nm excitation and 485 nm emission fluorescence mode on a Multimode Reader Cytation 5 (BioTek). The assay was carried out at QPS Austria GmbH (Grambach, Austria). Customized His tagged human full length G3BP2 (1-449) and the G3BP2 NTF2-like fragment (amino acids 1-139) were produced at GenScript Biotech.

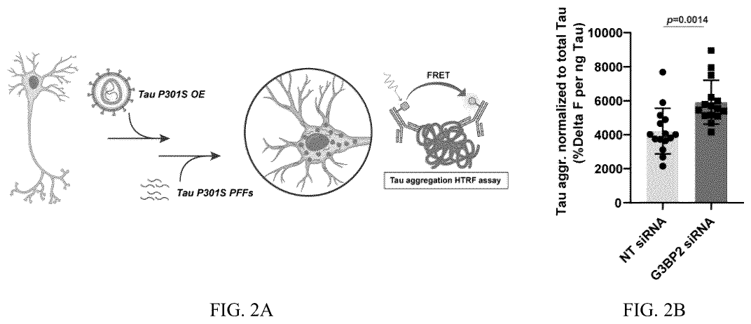

### FIGS. 2A and 2B:

iCell GlutaNeurons (FUJIFILM Cellular Dynamics, Cat. R1034) were seeded on 96-well plates at a density of 2×10⁵ cells/cm2-. Cells were treated with corresponding siRNAs on day 2, followed by transduction with lentiviral particles expressing P301S tau under an EF1a promoter at a multiplicity of infection (MOI) of 2 (Flash Therapeutics). On day 7, sonicated Tau pre-formed fibrils (PFFs) (StressMarq, Cat. SPR-471) were added to the media at a concentration of 1 μg/ml together with 0.2 μl Lipofectamine 2000 (Invitrogen, Cat. 11668019) per well. On day 14, Tau aggregates were measured using Tau aggregation assay kit (Cisbio, Cat. 6FTAUPEG) according to according to manufacturer's instructions. Total Tau content was determined via Tau AlphaLISA kit (Perkin Elmer, Cat.AL271).

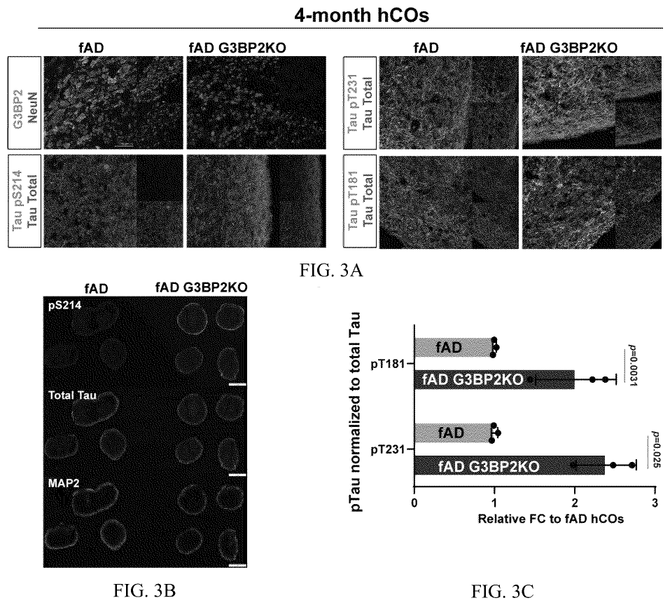

### FIGS. 3A-3C:

An fAD iPSC (SFC805-3-01) line was obtained from the StemBANCC consortium. To generate G3BP2 KO fAD iPSC, two sgRNAs, G3BP2_1 5′-UUGUAGGGCGGGAGUUUGUG-3′ (SEQ ID NO: 4) and G3BP2_2 5′-GUCGUUGUUCACGCACACG-3′ (SEQ ID NO: 5) (150 pmol/reaction) (Synthego, USA), and TrueCut Cas9 protein v2 (50 pmol/reaction) were used. hiPSC-derived hCOs were generated using a STEMdiff Cerebral Organoid Kit (STEMCELL Technologies, Cat. 08570) following the manufacturer's instructions.

For immunohistochemistry, hCOs were fixed using 4% paraformaldehyde (PFA) for 3 hours at RT, washed with PBS and immersed in 30% sucrose solution at 4° C. overnight. The PFA-fixed organoids were embedded in OCT compound (Sakura Finetek, Cat. 4583) and stored at −80° C. Organoid blocks were cut on a cryostat at 10 μm. For IHC, sections were permeabilized in 0.1% Triton X-100 and blocked with animal-free blocker (Vector Laboratories, Cat. SP-5030) for 30 min. Tissue sections were incubated with primary antibodies diluted in blocking solution for 1 h at RT, washed three times with PBS-T and further incubated with secondary antibodies for 1 h at RT. After washing with PBS-T, sections were incubated with PureBlu DAPI (Bio-Rad, Cat.1351303) for 3 min and mounted with Pro-Long Gold antifade mounting medium (Invitrogen, Cat. P36934). The following primary antibodies were used in IHC: G3BP2 (Novus, Cat. NBP1-82976), NeuN (Boster Bio, Cat. M11954-3), Tau pT231 (abeam, Cat.ab151559), Tau pT181 (abeam, Cat.ab254409) and Tau pS214 (abeam, Cat. ab170892). Species-specific Alexa Flour conjugated secondary antibodies were used in this study (Invitrogen). Fluorescence images were acquired with a SP8 confocal microscope (Leica, Germany) and an Olympus SLIDEVIEW VS200 (Olympus, Japan) slide scanner.

hCO lysates were prepared with CytoBuster protein extraction reagent (Millipore, Cat. 71009) supplemented with cOmplete EDTA-free protease inhibitor tablet (Roche, Cat. 11836170001) and PhosSTOP tablet (Roche, Cat. 4906845001). The levels of total tau were measured by human Tau AlphaLISA Detection Kit, and the levels of Tau pT231 and Tau pT181 were quantified using corresponding enzyme-linked immunosorbent assay (ELISA) kits (Thermo Fisher Scientific, Cat.KHB8051 and KH00631) following the manufacturer's instructions.

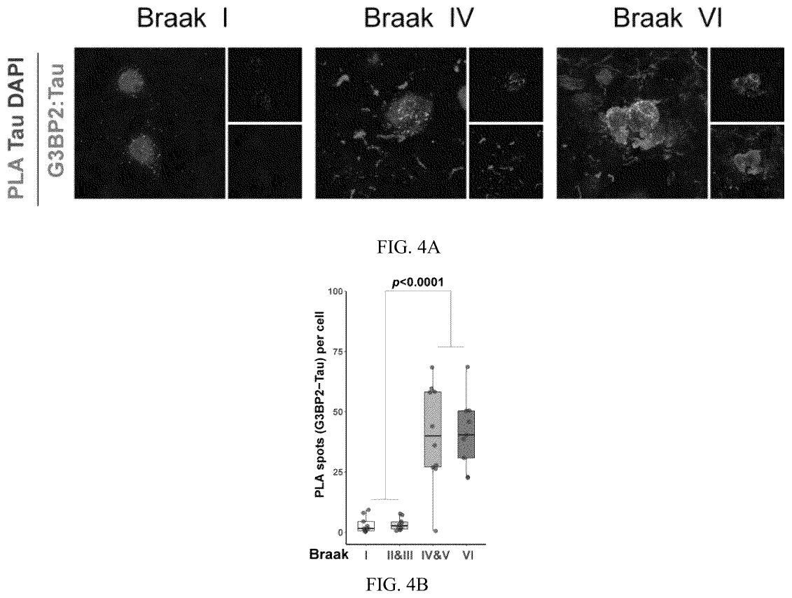

### FIGS. 4A and 4B:

The Duolink Proximity ligation assay (PLA) assay was performed following manufacturer's instructions. In brief, tissue sections were treated and incubated with antibodies as described for the immunohistochemisty. PLA was performed using the anti-rabbit PLUS (Sigma, Cat.DUO92002), anti-mouse MINUS (Sigma, Cat.DUO92004) probes and In Situ Detection Reagents-Green (Sigma, Cat.DUO92014). Following PLA, slides were incubated with PureBlu DAPI for 3 min and mounted with ProLong Gold antifade mounting medium. Fluorescence images were acquired with a SP8 confocal microscope. Olympus SLIDEVIEW VS200 slide scanner was used to scan images for quantification. The following primary antibodies were used in PLA: Tau HT7 (Thermo Fisher Scientific, Cat.MN1000), G3BP2 (Novus, Cat. NBP1-82976) and Tau polyclonal antibody (Novus, Cat. NBP2-25163).

For PLA quantification of brain sections, slices were scanned using an Olympus Slideview VS200 slide scanner, and more than 9000 cells were counted on average for each patient. Nuclei and PLA spots were quantified using Track Mate's LoG detector (ImageJ) with the same estimated blob diameter and threshold in each experiment. For each patient, the number of PLA spots was then normalized to the total number of cells.

1. A method for the treatment of a Tau associated disease, comprising administering to a subject an effective amount of a compound modulating a protein-protein interaction between a protein comprising a NTF2-like domain having the amino acid sequence set forth as SEQ ID NO: 3, or variants thereof, and Tau protein.

2. The method of claim 1, wherein the NTF2-like domain has at least 85% amino acid sequence identity with the amino acid sequence set forth as SEQ ID NO: 3.

3. The method of claim 1, wherein the NTF2-like domain has the amino acid sequence set forth as SEQ ID NO: 3.

4. The method of claim 1, wherein the compound promotes and/or enhances the protein—protein interaction between the protein comprising the NTF2-like domain and Tau protein.

5. The method of claim 1, wherein the compound is a mimetic of the protein—protein interaction between the protein comprising the NTF2-like domain and Tau protein.

6. The method of claim 1, wherein the Tau associated disease is selected from the group consisting of Alzheimer's Disease, amyotrophic lateral sclerosis, Parkinson's disease, Dementia pugilistica, Down's Syndrome, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotemporal dementia, frontotemporal dementia with parkinsonism linked to chromosome 17, Hallevorden-Spatz disease, Niemann-Pick disease type C, Pallido-ponto-nigral degeneration, Pick's disease, progressive subcortical gliosis, progressive supranuclear palsy, tangle-only dementia, postencephalitic Parkinsonism, and myotonic dystrophy.

7. The method of claim 1, wherein the protein comprising the NTF2-like domain is a G3PB2 protein.

8. The method of claim 1, wherein the protein comprising the NTF2-like domain and the Tau protein are human proteins.

9. A compound modulating the protein-protein interaction between a protein comprising a NTF2-like domain having an amino acid sequence set forth as SEQ ID NO: 3 or variants thereof and Tau protein, for use in the treatment of a Tau associated disease.

10. The compound for use of claim 9, wherein the NTF2-like domain has at least 85% amino acid sequence identity with the amino acid sequence set forth as SEQ ID NO: 3.

11. The compound for use of claim 9, wherein the NTF2-like domain has the amino acid sequence set forth as SEQ ID NO: 3.

12. The compound for use according to claim 9, wherein the Tau associated disease is selected from the group consisting of Alzheimer's Disease, amyotrophic lateral sclerosis, Parkinson's disease, Dementia pugilistica, Down's Syndrome, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotemporal dementia, frontotemporal dementia with parkinsonism linked to chromosome 17, Hallevorden-Spatz disease, Niemann-Pick disease type C, Pallido-ponto-nigral de-generation, Pick's disease, progressive subcortical gliosis, progressive supranuclear palsy, tangle-only dementia, postencephalitic Parkinsonism, and myotonic dystrophy.

13. A protein comprising a NTF2-like domain having an amino acid sequence set forth as SEQ ID NO: 3, or variants thereof, for use in the identification of a compound modulating the protein—protein interaction between the protein comprising the NTF2-like domain and Tau protein.

14. The protein for use of claim 13, wherein the NTF2-like domain has at least 85% amino acid sequence identity with the amino acid sequence set forth as SEQ ID NO: 3.

15. The protein for use of claim 13, wherein the NTF2-like domain has the amino acid sequence set forth as SEQ ID NO: 3.

16. The protein for use of claim 13, wherein the protein is human G3BP2 protein.

17. A protein comprising a NTF2-like domain having an amino acid sequence set forth as SEQ ID NO: 3, or variants thereof, for use as a target protein for the treatment of a Tau associated disease.

18. The protein for use of claim 17, wherein the NTF2-like domain has at least 85% amino acid sequence identity with the amino acid sequence set forth as SEQ ID NO: 3.

19. The protein for use of claim 17, wherein the NTF2-like domain has the amino acid sequence set forth as SEQ ID NO: 3.

20. The protein for use of claim 17, wherein the protein is human G3BP2 protein.


SEQUENCE LISTING (5 sequences)

SEQ ID NO: 1 - protein, 482 aa, Homo sapiens
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEERQPSPEPVQENANSGYYEAHPVTNGIEEPLEESSHEPEPEPESETKTEELKPQVEEKNLEELEEKSTTPPPAEPVSLPQEPPKAFSWASVTSKNLPPSGTVSSSGIPPHVKAPVSQPRVEAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 2 - protein, 449 aa, Homo sapiens
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEERQPSPEPVQENANSGYYEAHPVTNGIEEPLEESSHEPEPEPESETKTEELKPQVEEKNLEELEEKSTTPPPAEPVSLPQEPPKPRVEAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 3 - protein, 123 aa, Homo sapiens
VGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRY

SEQ ID NO: 4 - RNA, 20 nt, synthetic construct
  source 1..20: mol_type = other RNA
  misc_RNA 1..2: note = uracil
  misc_RNA 4: note = uracil
  misc_RNA 15..17: note = uracil
  misc_RNA 19: note = uracil
ttgtagggcgggagtttgtg

SEQ ID NO: 5 - RNA, 19 nt, synthetic construct
  source 1..19: mol_type = other RNA
  misc_RNA 2: note = uracil
  misc_RNA 5..6: note = uracil
  misc_RNA 8..9: note = uracil
gtcgttgttcacgcacacg